(19) United States
(12) Patent Application Publication
(10) Pub. No.: US 2026/0337232 A1
(43) Pub. Date: Oct. 29, 2026

(54) SOLID STATE FORMS OF RENCOFILSTAT

(71) Applicant: Achille Life Sciences Limited, London (GB)

(72) Inventors: Robert Thomas FOSTER, London (GB); Daniel Joseph TREPANIER, London (GB); Thomas Paul ZABAWA, London (GB)

(73) Assignee: Achille Life Sciences Limited, London (GB)

(21) Appl. No.: 19/491,037

(22) PCT Filed: Jun. 7, 2024
(86) PCT No.: PCT/US2024/033050

§ 371 Date: Dec. 8, 2025

Related U.S. Application Data
(60) Provisional application No. 63/471,879, filed on Jun. 8, 2023.

(51) Int. Cl. C07K 7/64; A61K 9/16; A61K 38/13

(52) U.S. Cl. CPC C07K 7/645; A61K 9/1682; A61K 38/13

(57) ABSTRACT

This invention relates to solid state forms of Rencofilstat (RCF), pharmaceutical compositions comprising the same, and methods of manufacturing pharmaceutical dosage forms utilizing the same.

### CROSS-REFERENCE TO RELATED APPLICATION

This application claims priority from U.S. Provisional Application Ser. No. 63/471,879, filed Jun. 8, 2023, the disclosure of which is incorporated by reference herein in its entirety.

### FIELD OF THE INVENTION

The present invention relates to solid state forms of Rencofilstat (RCF, also known as CRV431), processes for preparation thereof and pharmaceutical compositions thereof.

### BACKGROUND OF THE INVENTION

RCF is a small molecule cyclophilin inhibitor under clinical development for the treatment of liver diseases including liver fibrosis and hepatocellular carcinoma. In preclinical studies, RCF has shown anti-viral activity against a number of viruses including hepatitis B, hepatitis C, and HIV, and anti-fibrotic activity in the liver in a number of in vivo models. RCF (shown in FIG. 1B) is a derivative of cyclosporine A (CsA) (shown in FIG. 1A), a neutral cyclic peptide consisting of eleven amino acids, wherein amino acids 1 and 3 have been chemically modified as shown in FIG. 1B.

Compared to CsA, RCF desirably has reduced or no immunosuppressive activity, with improved binding for, and inhibition of, cyclophilin (CyP).

U.S. Pat. No. 9,200,038 and U.S. Patent Application Ser. No. 63/486,959, the disclosures of both of which are incorporated by reference herein in their entireties, describe, respectively, laboratory-scale and commercial-scale methods of producing RCF.

Polymorphism, the occurrence of different crystal forms, is a property of some molecules and molecular complexes. A single compound, like RCF, may give rise to a variety of polymorphs having distinct crystal structures and physical properties like melting point, thermal behaviors (e.g. measured by thermogravimetric analysis—“TGA”, or differential scanning calorimetry—“DSC”), powder X-ray diffraction (PXRD) pattern, infrared absorption fingerprint, Raman absorption fingerprint, and solid state (¹³C-) NMR spectrum. One or more of these techniques may be used to distinguish different polymorphic forms of a compound.

Different solid state forms (including solvated forms) of an active pharmaceutical ingredient may possess different properties. Such variations in the properties of different solid state forms and solvates may provide a basis for improving formulation, for example, by facilitating better processing or handling characteristics, improving the dissolution profile, or improving stability and shelf-life. These variations in the properties of different solid state forms may also provide improvements to the final dosage form, for instance, if these serve to improve bioavailability. Different solid state forms and solvates of an active pharmaceutical ingredient may also give rise to a variety of polymorphs or crystalline forms, which may in turn provide additional opportunities to use variations in the properties and characteristics of a solid active pharmaceutical ingredient for providing an improved product.

Discovering new solid state forms and solvates of a pharmaceutical product can provide materials having desirable processing properties, such as ease of handling, ease of processing, storage stability, and ease of purification or as desirable intermediate crystal forms that facilitate conversion to other polymorphic forms. New polymorphic forms and solvates of a pharmaceutically useful compound can also provide an opportunity to improve the performance characteristics of a pharmaceutical product (dissolution profile, bioavailability, etc.). This enlarges the repertoire of materials that a formulation scientist has available for formulation optimization, for example, by providing a product with different properties, e.g., a different crystal habit, higher crystallinity or polymorphic stability, which may offer better processing or handling characteristics, improved dissolution profile, or improved shelf-life.

For at least these reasons, there is a need for solid state forms (including solvated forms) of RCF.

### SUMMARY OF THE INVENTION

The present disclosure relates to solid state forms of RCF and to pharmaceutical compositions comprising these solid state forms.

In a first embodiment, the invention provides a solid state form of Rencofilstat designated as Form 1, characterized by data selected from one or more of the following:
(a) a powder X-ray diffraction (PXRD) pattern having the following peaks when measured at an angle of 2-theta±0.2 degrees 2-theta:

| | Position | | |
|---|---|---|---|
| # | 2θ Value | Intensity | Height |
| 1 | 4.7083 | 110408.4 | 506201.8 |
| 2 | 5.5849 | 4938.0 | 19820.2 |
| 3 | 6.9325 | 23830.6 | 97647.3 |
| 4 | 7.3994 | 31711.6 | 146608.3 |
| 5 | 8.6831 | 44562.8 | 179637.7 |
| 6 | 9.0191 | 24454.4 | 127945.9 |
| 7 | 9.5021 | 13383.6 | 73720.9 |
| 8 | 9.9659 | 32574.3 | 105011.0 |
| 9 | 10.2239 | 40704.6 | 224840.2 |
| 10 | 10.8055 | 4394.7 | 21434.1 |
| 11 | 11.2675 | 29302.0 | 128555.3 |
| 12 | 12.4411 | 20228.9 | 73141.2 |
| 13 | 13.3245 | 7920.7 | 45356.6 |
| 14 | 14.3006 | 19463.1 | 81718.0 |
| 15 | 14.7921 | 20015.4 | 99330.9 |
| 16 | 15.2066 | 5979.3 | 27102.5 |
| 17 | 15.7326 | 35689.7 | 142788.1 |
| 18 | 16.1205 | 30936.2 | 167994.0 |
| 19 | 16.9995 | 56657.4 | 182244.2 |
| 20 | 17.3235 | 13650.7 | 76633.3 |
| 21 | 17.6441 | 44849.0 | 208985.8 |
| 22 | 18.2004 | 2024.8 | 19622.8 |
| 23 | 18.9752 | 10743.3 | 49581.2 |
| 24 | 19.5894 | 52059.2 | 170752.0 |
| 25 | 20.0000 | 10473.1 | 43527.3 |
| 26 | 20.6846 | 21595.7 | 59901.1 |
| 27 | 21.1045 | 13355.2 | 48305.6 |
| 28 | 21.5370 | 9929.7 | 46979.1 |
| 29 | 21.9448 | 8187.3 | 38250.4 |
| 30 | 22.8054 | 51297.4 | 130002.2 |
| 31 | 24.0205 | 41704.5 | 153171.9 |
| 32 | 25.0603 | 29177.3 | 88303.9 |
| 33 | 25.6479 | 6381.3 | 26373.9 |
| 34 | 26.2237 | 8467.7 | 41999.4 |
| 35 | 27.0195 | 19906.9 | 74652.4 |
| 36 | 27.9666 | 19348.7 | 70994.9 |
| 37 | 29.4712 | 12866.7 | 55353.3 |
| 38 | 30.3381 | 6590.5 | 23877.9 |
| 39 | 31.0550 | 3514.6 | 12175.9 |
| 40 | 31.9574 | 11339.0 | 21506.4 |
| 41 | 32.7767 | 3029.2 | 12873.4 |
| 42 | 34.0085 | 9293.7 | 26166.1 |
| 43 | 35.1488 | 7014.5 | 34897.0 |
| 44 | 36.3905 | 3607.4 | 16735.8 |
| 45 | 36.9099 | 1433.2 | 9239.4 |
| 46 | 38.7824 | 4549.6 | 7702.9 |

(b) a PXRD pattern substantially as depicted in FIG. 2;
(c) a solid state ¹³C NMR spectrum having characteristic peaks at: 186.54, 184.52 and 181.38 ppm±0.2 ppm;
(d) a solid state ¹³C NMR spectrum having the following chemical shift absolute differences from reference peak at 86.00 ppm±1 ppm: 100.54, 98.52 and 95.38 ppm±0.1 ppm;
(e) a solid state ¹³C NMR spectrum having peaks at: 186.54, 184.52, 182.94, 181.38, 86.00, 71.69, 68.74, 66.22, 62.51, 61.58, 58.47, 56.13, 52.31, 51.22, 50.08, 48.39, 45.60, 44.20, 42.45, 41.66, 40.73, 39.52, 38.29, 36.28, 35.46, 34.41, 33.59, 32.69, 31.42, 30.66, 28.34, 26.44, and 22.38 ppm±0.2 ppm;
(f) a solid state ¹³C NMR spectrum substantially as depicted in FIG. 5;
(g) an FT-IR spectrum having peaks at: 533.72, 583.13, 632.43, 781.53, 864.27, 942.13, 1002.76, 1031.85, 1086.67, 1126.54, 1222.31, 1269.69, 1305.09, 1388.37, 1407.96, 1463.79, 1544.55, 1623.19, 1654.41, 1674.28, 1686.39, 2871.76, 2926.28, 2956.64, 3296.78, 3333.48, 3430.73, and 3477.32 cm⁻¹±4 cm⁻¹; and
(h) a combination of any two or more of (a)-(g).

In an additional embodiment, the solid state form of Rencofilstat designated as Form 1 is characterized by a PXRD pattern having peaks at: 4.7083, 8.6831, 10.2239, 16.9995 and 17.6441 degrees 2-theta±0.2 degrees 2-theta, and also having any one, two, three, four or five additional peaks at: 7.3994, 15.7326, 16.1205, 19.5894 and 24.0205 degrees 2-theta±0.2 degrees 2-theta.

In a further embodiment, the invention provides a solid state form of Rencofilstat designated as Form 2, characterized by data selected from one or more of the following:
(a) a PXRD pattern having the following peaks when measured at an angle of 2-theta±0.2 degrees 2-theta:

| | Position | | |
|---|---|---|---|
| # | 2θ Value | Intensity | Height |
| 1 | 4.6736 | 19955.5 | 99826.7 |
| 2 | 5.8747 | 8398.2 | 38897.8 |
| 3 | 7.6125 | 14330.3 | 80845.4 |
| 4 | 7.9550 | 16095.2 | 101606.1 |
| 5 | 8.4823 | 5571.0 | 29324.1 |
| 6 | 9.4833 | 36325.4 | 183229.2 |
| 7 | 10.6611 | 7462.2 | 36808.3 |
| 8 | 11.8725 | 22234.4 | 109115.8 |
| 9 | 13.5826 | 3056.5 | 14816.2 |
| 10 | 14.9173 | 4586.7 | 29826.3 |
| 11 | 15.2227 | 11555.9 | 68004.3 |
| 12 | 16.0121 | 3415.7 | 19811.9 |
| 13 | 16.3762 | 2050.1 | 12136.3 |
| 14 | 16.8084 | 5000.0 | 20991.0 |
| 15 | 17.5943 | 19701.4 | 96090.9 |
| 16 | 18.1623 | 9297.5 | 38272.9 |
| 17 | 18.7169 | 3070.0 | 20037.7 |
| 18 | 19.1112 | 6916.9 | 37519.0 |
| 19 | 20.2712 | 4754.8 | 25291.7 |
| 20 | 20.4733 | 2900.9 | 20181.2 |
| 21 | 20.8856 | 3097.6 | 16085.0 |
| 22 | 21.9116 | 6018.9 | 14049.2 |
| 23 | 22.5662 | 5268.3 | 13773.0 |
| 24 | 23.5071 | 3218.8 | 11820.8 |
| 25 | 23.8611 | 5452.1 | 25641.9 |
| 26 | 24.4246 | 3720.4 | 17285.2 |
| 27 | 25.1597 | 6319.7 | 20122.7 |
| 28 | 26.5595 | 2034.6 | 11220.0 |
| 29 | 28.3775 | 1982.9 | 7554.1 |
| 30 | 29.3789 | 1892.3 | 6813.8 |
| 31 | 30.3225 | 2388.9 | 10877.8 |
| 32 | 31.2591 | 3454.6 | 8665.8 |
| 33 | 34.1043 | 3992.9 | 8491.2 |
| 34 | 36.1792 | 977.9 | 2828.7 |

(b) a PXRD pattern substantially as depicted in FIG. 3;
(c) a solid state ¹³C NMR spectrum having characteristic peaks at: 187.29, 184.49, 182.82 and 181.68 ppm±0.2 ppm;
(d) a solid state ¹³C NMR spectrum having the following chemical shift absolute differences from reference peak at 85.81 ppm #1 ppm: 101.48, 98.68, 97.01 and 95.87 ppm±0.1 ppm;
(e) a solid state ¹³C NMR spectrum having peaks at: 187.29, 185.80, 184.49, 183.51, 182.82, 181.68, 85.81, 79.48, 71.66, 69.64, 67.97, 66.02, 64.79, 62.20, 61.35, 59.89, 55.78, 52.15, 49.64, 47.18, 45.69, 44.04, 42.38, 41.19, 40.21, 38.88, 36.00, 34.79, 34.06, 33.12, 32.35, 30.82, 30.00, 28.50, 25.91, 25.09, and 22.40 ppm±0.2 ppm;
(f) a solid state ¹³C NMR spectrum substantially as depicted in FIG. 6;
(g) an FT-IR spectrum having peaks at: 536.28, 571.41, 596.14, 645.23, 769.57, 859.78, 942.97, 1090.73, 1263.66, 1381.97, 1411.63, 1466.98, 1536.14, 1621.07, 1676.31, 1721.79, 1737.08, 2871.91, 2932.36, 2957.01, 3301.60, and 3487.45 cm⁻¹±4 cm⁻¹; and
(h) a combination of any two or more of (a)-(g).

In another embodiment, the solid state form of Rencofilstat designated as Form 2 is characterized by a PXRD pattern having peaks at: 4.6736, 7.9550, 9.4833, 11.8725 and 17.5943 degrees 2-theta±0.2 degrees 2-theta, and also having any one, two, three, four or five additional peaks at: 5.8747, 7.6125, 15.2227, 18.1623, and 19.1112 degrees 2-theta±0.2 degrees 2-theta.

In yet another embodiment, the present disclosure encompasses pharmaceutical formulations comprising the above described solid state forms of RCF and at least one pharmaceutically acceptable excipient, preferably for oral administration in dosage forms such as tablets, capsules etc.

In a further embodiment, the present invention provides methods for manufacturing a pharmaceutical dosage form based on a self-microemulsifying drug delivery system (SMEDDS) or a spray dried dispersion.

In a preferred embodiment, the inventive SMEDDS dosage form is prepared by solubilizing crystalline (preferably Form 1) RCF in a mixture of Vitamin E, glyceryl monolinoleate, propylene glycol, diethylene glycol monoethyl ether, ethanol, and polyoxyl castor oil, wherein the Vitamin E, glyceryl monolinoleate, propylene glycol, diethylene glycol monoethyl ether, ethanol, and polyoxyl castor oil are at a weight ratio, respectively, of about (0.75-1.5)/(0.5-2)/(2-5)/(2-5)/(2-2.4)/(4-8).

In another preferred embodiment, the inventive spray dried dispersion dosage form is prepared by mixing crystalline (preferably Form 1) RCF with a pharmaceutically acceptable polymer; dissolving the mixture in an organic solvent; forming liquid droplets of the dissolved mixture; spraying the liquid droplets onto a receiving surface; and then drying the sprayed mixture to remove residual solvent.

### BRIEF DESCRIPTION OF THE DRAWINGS

FIG. 1A shows the chemical structure of cyclosporine A.

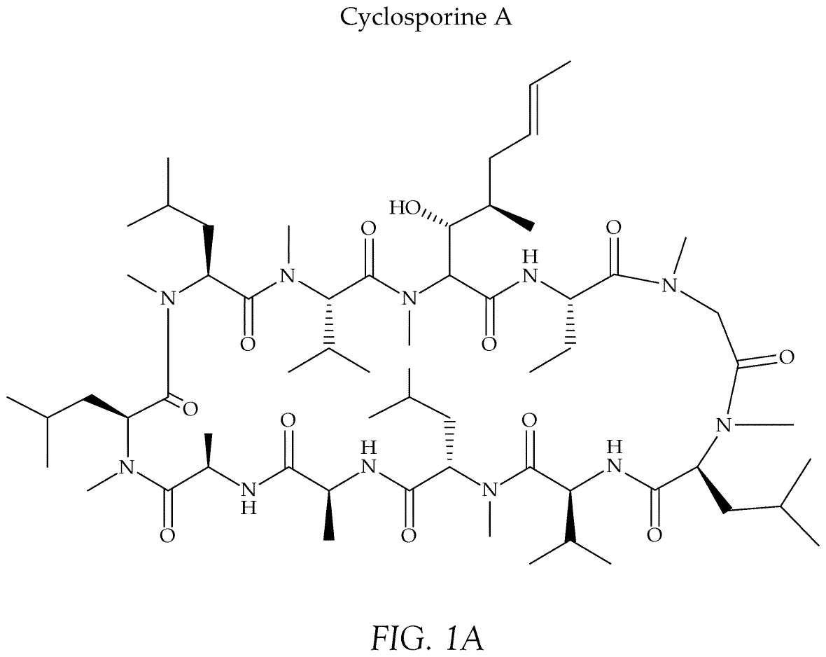

FIG. 1B shows the chemical structure of CRV431 (RCF).

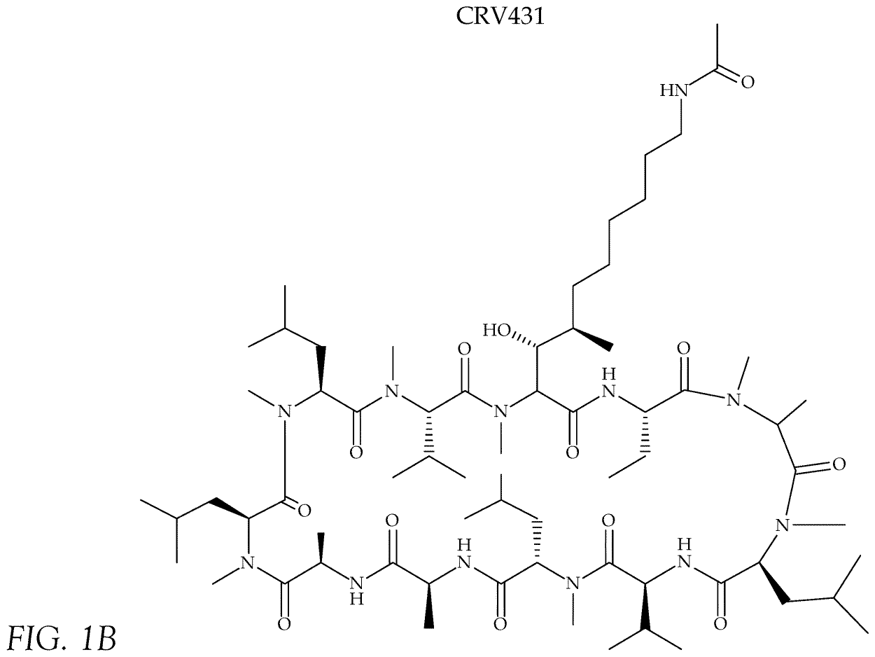

FIG. 2 shows a powder X-ray diffraction (“powder XRD” or “PXRD”) pattern of RCF Form 1.

FIG. 3 shows a PXRD pattern of RCF Form 2.

FIG. 4 shows FT-IR spectra of RCF Forms 1 and 2, respectively.

FIG. 5 shows a solid state ¹³C NMR spectrum (range from 200-0 ppm) of RCF Form 1.

FIG. 6 shows a solid state ¹³C NMR spectrum (range from 200-0 ppm) of RCF Form 2.

### DETAILED DESCRIPTION

Disclosed herein are two solid state forms of RCF. Form 1 is an anhydrate with a melting point of about 230 °C to 233 °C and a heat of fusion of 72.3 J/g.

Form 2 appears to be an isomorphic, non-stoichiometric solvate with a melting point between 13° and 145 °C depending on its composition. Form 2 can be stabilized by, e.g., isopropyl acetate (iProAc), 1,4-dioxane, 2-propanol, methyl tert-butyl ether, toluene, benzyl alcohol, or diethylamine. Form 2 does not exist as a stable hydrate, though it is moderately hygroscopic. Critically, Form 2 is not stabilized by acetonitrile (ACN) or methyl isobutyl ketone (MIBK).

Form 2 converts to Form 1 upon slurry in ACN, ACN/water blends, or upon heating above 150 °C. Form 2 melts at about 137 °C, followed by recrystallization to Form 1 at about 147 °C. This recrystallization event is followed by melting of Form 1 at around 230 °C.

### DEFINITIONS

A crystal form may be referred to herein as being characterized by graphical data “as depicted in” or “as shown in” a Figure. Such data include, for example, powder X-ray diffractograms and solid state NMR spectra. As is well-known in the art, the graphical data potentially provide additional technical information to further define the respective solid state form (a so-called “fingerprint”), which can not necessarily be described by reference to numerical values or peak positions alone. In any event, the skilled person will understand that such graphical representations of data may be subject to small variations, e.g., in peak relative intensities and peak positions due to factors such as variations in instrument response and variations in sample concentration and purity, which are well known to the skilled person. Nonetheless, the skilled person would readily be capable of comparing the graphical data in the Figures herein with graphical data generated for an unknown crystal form and confirming whether the respective sets of graphical data characterize the same crystal form or two different crystal forms. Thus, a crystal form of RCF referred to herein as being characterized by graphical data “as depicted in” or “as shown in” a Figure will thus be understood to include any crystal forms of RCF characterized with the graphical data having such small variations, as are well known to the skilled person, in comparison with the Figure.

A solid state form (or polymorph) may be referred to herein as polymorphically pure or substantially free of any other solid state (or polymorphic) forms. As used herein in this context, the expression “substantially free of any other forms” will be understood to mean that the solid state form contains about 20% or less, about 10% or less, about 5% or less, about 2% or less, about 1% or less, or about 0% of any other forms of the subject compound as measured, for example, by PXRD.

Thus, solid state forms of RCF described herein as substantially free of any other solid state forms would be understood to contain greater than about 80% (w/w), greater than about 90% (w/w), greater than about 95% (w/w), greater than about 98% (w/w), greater than about 99% (w/w), or about 100% (w/w) of the subject solid state form of RCF. Accordingly, in some embodiments of the disclosure, the described solid state forms of RCF may contain from about 1% to about 20% (w/w), from about 5% to about 20% (w/w), or from about 5% to about 10% (w/w) of one or more other solid state forms of RCF.

As used herein, unless stated otherwise, PXRD peaks reported herein may be measured using CuK_{α} radiation, λ=1.5418 Å.

As used herein, percentages are weight-percent (wt %) unless otherwise indicated.

A process or step may be referred to herein as being carried out “overnight.” This refers to a time interval, e.g., for the process or step, that spans the time during the night, when that process or step may not be actively observed. This time interval is from about 8 to about 20 hours, or about 10 to about 18 hours, typically about 16 hours.

The use of various solvents is described herein, often designated by the respective abbreviations shown in the following Table 1:

| Solvent | Shorthand |
|---|---|
| 1,4-dioxane | DX |
| 2-propanol | IPA |
| acetic acid | AcOH |
| acetone | Ace |
| acetonitrile | ACN |
| benzyl alcohol | BnOH |
| Cremophor RH40 | RH40 |
| dichloromethane | DCM |
| diethylamine | DEA |
| diethylene glycol | DEGMEE |
| monoethyl ether | |
| diisopropyl ether | DIPE |
| dimethylformamide | DMF |
| dimethylsulfoxide | DMSO |
| ethanol | EtOH |
| ethyl acetate | EtOAc |
| glycerol monolinoleate | GML |
| heptane | Hep |
| isopropyl acetate | iProAc |
| methanol | MeOH |
| methyl ethyl ketone | MEK |
| methyl isobutyl ketone | MIbK |
| methyl tert-butyl ether | MTBE |
| propylene glycol | PG |
| tetrahydrofuran | THF |
| toluene | Tol |
| vitamin E | VitE |
| water | H₂O |

The amount of a given solvent employed in a chemical process, e.g., a reaction or a crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding methyl tert-butyl ether (“MTBE”) (1.5 v/v) to a 100 ml reaction mixture would indicate that 150 mL of MTBE was added.

As used herein, the term “reduced pressure” refers to a pressure of about 10 mbar to about 50 mbar.

### Pharmaceutical Formulations

The present disclosure also provides the use of a solid state form of RCF for preparing other solid state forms of RCF.

The present invention further contemplates the use of the above-described solid state forms of RCF, individually or in combination, for the preparation of pharmaceutical compositions and/or formulations, preferably oral formulations, e.g. tablets or capsules. Accordingly, the present disclosure encompasses pharmaceutical formulations comprising at least one of the above described solid state forms of RCF, or combinations thereof, and at least one pharmaceutically acceptable excipient.

Pharmaceutically acceptable excipients may be added to the formulations of the present invention for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include, for example, acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include, for example, alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®), and starch.

Glidants can be added to improve the flowability of a non-compacted solid composition and to improve the accuracy of dosing. Excipients that can function as glidants include, for example, colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. However, a lubricant can be added to the composition to reduce adhesion and ease the release of the product from the dye. Suitable lubricants include, for example, magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include, for example, maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present disclosure, the active ingredient and any other solid excipients may be dissolved or suspended in a liquid carrier such as water, vegetable oil, alcohol, polyethylene glycol, propylene glycol, or glycerin.

Liquid pharmaceutical compositions also may contain emulsifying agents to disperse uniformly throughout the composition an active ingredient or other excipient that is not soluble in the liquid carrier. Emulsifying agents that can be useful in liquid compositions of the present disclosure include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present disclosure can also contain a viscosity enhancing agent to improve the mouth-feel of the product and/or coat the lining of the gastrointestinal tract. Such agents may include, for example, acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as sorbitol, saccharin, sodium saccharin, sucrose, aspartame, fructose, mannitol, and invert sugar also can be added to improve taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate.

Selection of excipients and the amounts used can be readily determined by the formulation scientist based upon experience and consideration of standard procedures and reference works in the field, such as Ansel et al., Pharmaceutical Dosage Forms and Drug Delivery Systems, 7th ed., the disclosure of which hereby is incorporated by reference herein in its entirety.

### Dosages

The solid compositions of the present disclosure may include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in a preferred embodiment the route of administration is oral.

The dosages can be conveniently presented in unit dosage form and prepared by any of the methods well-known in the pharmaceutical arts.

Dosage forms may include solid dosage forms such as tablets, powders, capsules, suppositories, sachets, troches, and lozenges, as well as liquid syrups, suspensions, and elixirs.

The dosage form of the present disclosure can be a capsule comprising a predetermined amount of a composition disclosed herein; as a powder or granules; as a solution or a suspension in an aqueous liquid or a non-aqueous liquid, contained within either a hard or soft shell. The shell can be made from gelatin and optionally may contain a plasticizer such as glycerin, and optionally sorbitol, an opacifying agent and/or colorant.

A composition for tableting or capsule filling can be prepared by wet granulation. In wet granulation, some or all of the active ingredients and excipients in powder form are blended and then further mixed in the presence of a liquid, typically water, which causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the active pharmaceutical ingredients and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include, for example, microcrystalline cellulose, spray dried lactose, dicalcium phosphate dihydrate, and colloidal silica.

A capsule filling of the present invention can comprise any of the aforementioned blends and granulates that were described with reference to tableting, but these are not subjected to a final tableting step.

A pharmaceutical formulation as contemplated herein may be formulated for administration to a mammal, preferably a human. The compositions disclosed herein can be formulated, for example, as a viscous liquid solution or suspension, or a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include, for example, alcohol USP, benzyl alcohol NF, benzyl benzoate USP, and Castor oil USP. Additional substances can be added to the formulation such as buffers, solubilizers, and antioxidants, among others.

The invention is further illustrated by reference to the following examples describing in detail the preparation and analysis of the compositions of the invention.

### Methods of Manufacturing Preferred Dosage Forms

The present invention also encompasses methods for manufacturing a pharmaceutical dosage form based on a self-microemulsifying drug delivery system (SMEDDS) or a spray dried dispersion.

SMEDDS (self-microemulsifying drug delivery systems) are homogeneous mixtures of hydrophilic surfactant, co-solvents and oil that form an oil-in-water (o/w) micro-emulsion quickly after gentle agitation and dilution in an aqueous medium. This type of supersaturated environment has been shown to improve drug solubility and absorption.

A SMEDDS dosage form of the invention is prepared by solubilizing crystalline (preferably Form 1) RCF in a mixture of Vitamin E, glyceryl monolinoleate, propylene glycol, diethylene glycol monoethyl ether, ethanol, and polyoxyl castor oil, wherein the Vitamin E, glyceryl monolinoleate, propylene glycol, diethylene glycol monoethyl ether, ethanol, and polyoxyl castor oil are at a weight ratio, respectively, of about (0.75-1.5)/(0.5-2)/(2-5)/(2-5)/(2-2.4)/(4-8).

Spray drying co-precipitates a drug molecule and a polymer carrier in a stable amorphous solid dispersion, resulting in improved dissolution rates and enhanced bioavailability of poorly soluble compounds. A spray-dried dispersion (SDD) places a drug molecule in a polymer-mediated, solubilized state and maintains that amorphous state during transition through the patient's gastrointestinal (GI) tract. SDDs can be prepared from a solution wherein both the drug molecule and a pharmaceutically acceptable polymer are dissolved in a suitable solvent that can be readily evaporated (including, without limitation, acetone, methanol, methanol/water, or acetone/water mixtures). Pharmaceutically acceptable polymers include, without limitation, hydroxypropyl methylcellulose (HPMC), hydroxypropyl methylcellulose acetate succinate (HPMC-AS), and polyvinylpyrrolidone (PVP).

In preparing an SDD, both the drug molecule and the polymer must be completely dissolved, in order to avoid the inclusion of crystalline residues of the drug molecule in the final SDD. The resulting solution is then delivered into a spray drying unit, which quickly converts the solution into droplets. These droplets dry rapidly in-flight as they travel through, and from the exit of, the spray dryer to a receiving surface (which may include a three-dimensional shell or mold). The solvent's rapid evaporation traps the drug-polymer mixture in the amorphous state and produces a low-density solid particle. The dried SDD material then goes through a post-drying step to ensure complete removal of the solvent.

An SDD dosage form of the invention is prepared by mixing crystalline (preferably Form 1) RCF with a pharmaceutically acceptable polymer; dissolving the mixture in an organic solvent; forming liquid droplets of the dissolved mixture; spraying the liquid droplets onto a receiving surface; and then drying the sprayed mixture to remove residual solvent. Preferably the polymer is HPMC. Preferred solvents are methanol or a methanol/water mixture.

### EXAMPLES

### Analytical Methods Used in the Examples Below

### Thermogravimetric Analysis (“TGA”)

Initial: Sample was placed in pre-tared 40 μL aluminum pans and loaded onto the autosampler of a TA Discovery TGA. Samples were heated at 20 °C/min to 300 °C.

Isolated from Slurry: Samples were placed in pre-tared 40 μL aluminum pans and loaded onto the autosampler of a TA Discovery TGA. Samples were heated at 3 °C/min to 100 °C and then 20 °C/min to 300 °C.

Recovered from saturated solutions: Cooled samples followed the method described for “isolated from slurry” samples. The samples recovered by evaporation were run at 10 °C to 300 °C.

### Differential Scanning Calorimetry (“DSC”)

Approximately 3 mg of sample was placed in a non-hermetic aluminum pan and analyzed on a Mettler Toledo DSC that was calibrated using indium (temperature) and sapphire (heat capacity). Samples were analyzed using STARe software.

RCF Form 1 sample was heated at 10 °C/min to 255 °C, cooled at 10 °C/min to 0 °C, and finally re-heated at 10 °C/min to 255 °C.

The isolated slurry solids and the cooled saturated solution solids were run with the same method, although only the first heat cycle was analyzed. The solids recovered by evaporation were only run through one heating at 10 °C/min, 0-255 °C.

### Example 1: Initial Characterization of RCF Form 1

Differential Scanning calorimetry (DSC): A sample of RCF was heated, and was determined to have a melting point of 231.2 °C and a heat of fusion of 72.3 J/g (94.2 kJ/mol). On cooling, a small exotherm is observed at 61.1 °C (−0.63 J/g). During the second heating cycle, a T_{g} was observed at 116.5 °C (ΔCₚ=0.52 J g⁻¹ K⁻¹) with no additional thermal events.

Thermogravimetric Analysis (TGA): RCF Form 1 did not lose any mass through the melt, consistent with an essentially solvent-free anhydrate. It began to decompose at 262 °C.

Dynamic vapor sorption (DVS): On desorption, 0.5% mass was lost between 95 and 0% RH (relative humidity), indicating that RCF Form 1 is a non-hygroscopic crystalline structure that does not readily form a hydrate from the solid state at ambient temperature. The initial sorption isotherm indicated a 10% mass loss between 50 and 60% RH, but post-DVS characterization and images acquired during sorption indicate no changes to the material, so this was likely an artifact of the experiment. NMR (as discussed further below), before and after DVS, matched with no indication of loss of any peaks or changes in relative intensity, suggesting that Form 1 is unchanged after exposure to 95% humidity followed by drying.

### Example 2: Initial Characterization of RCF Form 2

### Crystallization

Slurries of RCF Form 1 were prepared in 20 solvents or solvent blends. Slurries were held at 0, 25, or 50 °C with stirring for two weeks. In 12 of the 20 slurries, the solids at the end of the experiment were consistent with a different polymorph (Form 2). In the other 8 slurries, the final solids were consistent with the starting material (Form 1). These results are summarized in the following Table 2:

| FORM 1 RETAINED | | CONVERTED TO FORM 2 | |
|---|---|---|---|
| ACN | MIBK | 1,4-dioxane | IPA |
| DEGMEE | propylene glycol | iProAc | MTBE |
| Vitamin E | 80/20 DIPE/ACN | toluene | 80/20 iProAc/ACN |
| 50/50 iProAc/ACN | 70/30 iProAc/DCM | 70/30 DIPE/BnOH | 70/30 water/DEA |
| 80/20 heptane/THF | 80/20 water/AcOH | | |
| 50/50 MeOH/water | 70/30 water/glycerol | | |

RCF Form 1 did not convert to Form 2 in acetonitrile or in any of the formulation solvents tested (DEGMEE, propylene glycol, vitamin E). Form 2 formed in the pure solvents 1,4-dioxane, IPA, iProAc, MTBE, and toluene.

Most RCF Form 2 samples exhibited an endotherm and exotherm at ca. 127 and 145 °C, respectively. The only exceptions to this were the solids isolated from 30/70 DIPE/BnOH (no apparent transition in this temperature range) and from iProAc slurried at 0 °C (exotherm only). For two samples (from iProAc and MTBE at 25 °C), the exotherm occurs at slightly higher temperature. All samples melted at 230 °C. The DSC is consistent with that reported previously for Form 1. This is consistent with Form 2 having a melting point at ca. 137 °C and Form 1 having a melting point at 230 °C.

When RCF Form 2 melts, it appears to re-crystallize to Form 1 during or soon after the melting process. By contrast, after RCF Form 1 melts, no re-crystallization is observed during cooling or re-heating of the sample at the same scan rate. This suggests that Form 2 crystals seed crystallization of Form 1 from the melt and/or that Form 1 and Form 2 are similar crystal structures and, as solvent is lost from the Form 2 structure, it converts to Form 1.

All isolated solids consistent with Form 2 lose appreciable mass during heating, 1.8-5.5 wt % (0.27-0.7 mol ratio) between ambient temperature and 230 °C during TGA. In most cases, the mass loss is gradual and there is no indication of a sudden mass loss in the transition temperature range of 130-160 °C observed in DSC. All solids had been vacuum dried overnight at 40 °C prior to TGA.

Taken together, these data suggest that RCF Form 2 is likely a solvate that can accommodate many different solvents and is stable over a wide range of solvents and conditions between 0 and 50 °C.

### Example 3: RCF Form 2 Solvate Investigation

### Solvent Loss by Heating

To find further evidence of whether RCF Form 2 is a solvate, a Form 2 sample was heated to 70 °C for 3 days in a vacuum oven to drive off solvent. This process removed about half of the solvent and led to a more disordered Form 2. This suggests that Form 2 is not stable in the absence of solvent and that solvent is not easily removed from the crystal.

### Form 2 DVS

RCF Form 2 is moderately hygroscopic and reversibly sorbs and desorbs water at 25 °C up to 8.9 wt % water at 90% RH. This amount of water is equivalent to a 7:1 molar ratio of water to RCF. This is consistent with Form 2 being a non-stoichiometric solvate that can incorporate water into its open crystal structure.

### Form 2 Stability in High Water Activity Solvent Blend

In order to understand if RCF Form 2 can exist as a stable hydrate, Form 2 was slurried in saturated ACN/water blends with high water activity for 2 days. Form 2 was added to ACN/water blends (70-90% (w/w) water) previously saturated with Form 1. (Samples containing Form 1 solids had been heated to 70 °C overnight and then stirred at ambient temperature for 2 days. Remaining solids were still of Form 1, and these solids were removed via filtration through a 0.45 micron PTFE syringe filter.) After stirring these suspensions for 2 days, all remaining solids were of Form 1. For blends containing 70-74% (w/w) water, the Form 2 solids initially dissolved, and two liquid phases formed (liquid-liquid phase separation, LLPS), and solids were observed after 2 days stirring. For 90% (w/w) water, no dissolution of solids was observed.

The water activity in these experiments is between 0.88 and 0.94. The conversion of Form 2 solids to Form 1 solids at such high water activity confirms that while Form 2 can accommodate a large amount of water (as determined by DVS), it is not a stable hydrate.

### Example 4: PXRD Analysis of RCF Forms 1 and 2

For purposes of PXRD analysis, Form 1 and Form 2 solids were either loaded onto a zero background (high index Si crystal) holder (0.2 mm deep) or dried directly on a zero background holder by drop casting a slurry onto the holder. Samples were analyzed on a Rigaku MiniFlex 600 at 40 kV and 15 mA with a D/tex Ultra 1D detector.

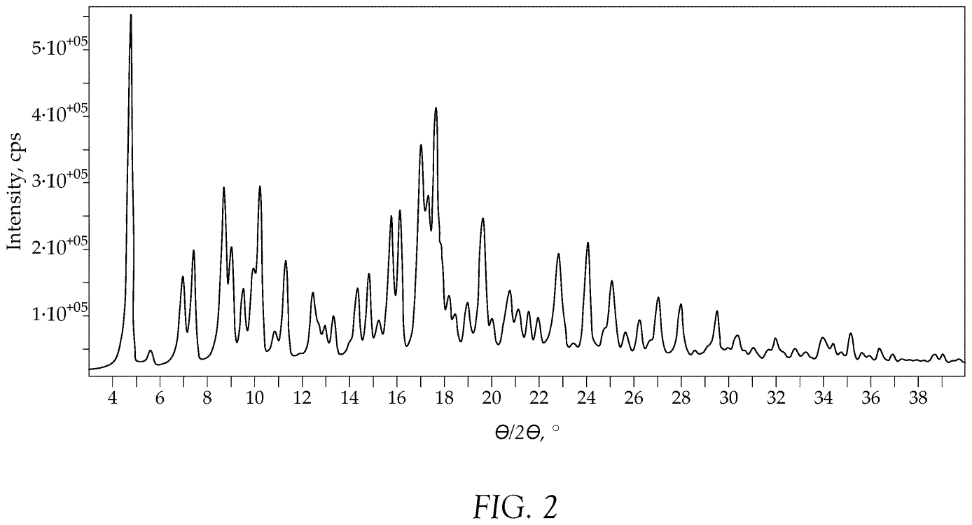

The PXRD pattern for Form 1 is depicted in FIG. 2, and has the following peaks when measured at an angle of 2-theta±0.2 degrees 2-theta:

| | Position | | |
|---|---|---|---|
| # | 2θ Value | Intensity | Height |
| 1 | 4.7083 | 110408.4 | 506201.8 |
| 2 | 5.5849 | 4938.0 | 19820.2 |
| 3 | 6.9325 | 23830.6 | 97647.3 |
| 4 | 7.3994 | 31711.6 | 146608.3 |
| 5 | 8.6831 | 44562.8 | 179637.7 |
| 6 | 9.0191 | 24454.4 | 127945.9 |
| 7 | 9.5021 | 13383.6 | 73720.9 |
| 8 | 9.9659 | 32574.3 | 105011.0 |
| 9 | 10.2239 | 40704.6 | 224840.2 |
| 10 | 10.8055 | 4394.7 | 21434.1 |
| 11 | 11.2675 | 29302.0 | 128555.3 |
| 12 | 12.4411 | 20228.9 | 73141.2 |
| 13 | 13.3245 | 7920.7 | 45356.6 |
| 14 | 14.3006 | 19463.1 | 81718.0 |
| 15 | 14.7921 | 20015.4 | 99330.9 |
| 16 | 15.2066 | 5979.3 | 27102.5 |
| 17 | 15.7326 | 35689.7 | 142788.1 |
| 18 | 16.1205 | 30936.2 | 167994.0 |
| 19 | 16.9995 | 56657.4 | 182244.2 |
| 20 | 17.3235 | 13650.7 | 76633.3 |
| 21 | 17.6441 | 44849.0 | 208985.8 |
| 22 | 18.2004 | 2024.8 | 19622.8 |
| 23 | 18.9752 | 10743.3 | 49581.2 |
| 24 | 19.5894 | 52059.2 | 170752.0 |
| 25 | 20.0000 | 10473.1 | 43527.3 |
| 26 | 20.6846 | 21595.7 | 59901.1 |
| 27 | 21.1045 | 13355.2 | 48305.6 |
| 28 | 21.5370 | 9929.7 | 46979.1 |
| 29 | 21.9448 | 8187.3 | 38250.4 |
| 30 | 22.8054 | 51297.4 | 130002.2 |
| 31 | 24.0205 | 41704.5 | 153171.9 |
| 32 | 25.0603 | 29177.3 | 88303.9 |
| 33 | 25.6479 | 6381.3 | 26373.9 |
| 34 | 26.2237 | 8467.7 | 41999.4 |
| 35 | 27.0195 | 19906.9 | 74652.4 |
| 36 | 27.9666 | 19348.7 | 70994.9 |
| 37 | 29.4712 | 12866.7 | 55353.3 |
| 38 | 30.3381 | 6590.5 | 23877.9 |
| 39 | 31.0550 | 3514.6 | 12175.9 |
| 40 | 31.9574 | 11339.0 | 21506.4 |
| 41 | 32.7767 | 3029.2 | 12873.4 |
| 42 | 34.0085 | 9293.7 | 26166.1 |
| 43 | 35.1488 | 7014.5 | 34897.0 |
| 44 | 36.3905 | 3607.4 | 16735.8 |
| 45 | 36.9099 | 1433.2 | 9239.4 |
| 46 | 38.7824 | 4549.6 | 7702.9 |

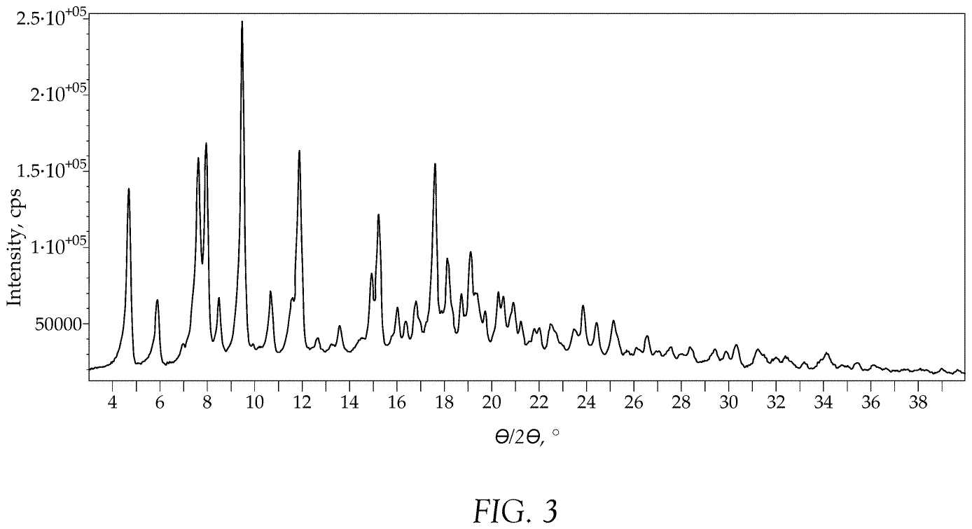

The PXRD pattern for Form 2 is depicted in FIG. 3, and has the following peaks when measured at an angle of 2-theta±0.2 degrees 2-theta:

| | Position | | |
|---|---|---|---|
| # | 2θ Value | Intensity | Height |
| 1 | 4.6736 | 19955.5 | 99826.7 |
| 2 | 5.8747 | 8398.2 | 38897.8 |
| 3 | 7.6125 | 14330.3 | 80845.4 |
| 4 | 7.9550 | 16095.2 | 101606.1 |
| 5 | 8.4823 | 5571.0 | 29324.1 |
| 6 | 9.4833 | 36325.4 | 183229.2 |
| 7 | 10.6611 | 7462.2 | 36808.3 |
| 8 | 11.8725 | 22234.4 | 109115.8 |
| 9 | 13.5826 | 3056.5 | 14816.2 |
| 10 | 14.9173 | 4586.7 | 29826.3 |
| 11 | 15.2227 | 11555.9 | 68004.3 |
| 12 | 16.0121 | 3415.7 | 19811.9 |
| 13 | 16.3762 | 2050.1 | 12136.3 |
| 14 | 16.8084 | 5000.0 | 20991.0 |
| 15 | 17.5943 | 19701.4 | 96090.9 |
| 16 | 18.1623 | 9297.5 | 38272.9 |
| 17 | 18.7169 | 3070.0 | 20037.7 |
| 18 | 19.1112 | 6916.9 | 37519.0 |
| 19 | 20.2712 | 4754.8 | 25291.7 |
| 20 | 20.4733 | 2900.9 | 20181.2 |
| 21 | 20.8856 | 3097.6 | 16085.0 |
| 22 | 21.9116 | 6018.9 | 14049.2 |
| 23 | 22.5662 | 5268.3 | 13773.0 |
| 24 | 23.5071 | 3218.8 | 11820.8 |
| 25 | 23.8611 | 5452.1 | 25641.9 |
| 26 | 24.4246 | 3720.4 | 17285.2 |
| 27 | 25.1597 | 6319.7 | 20122.7 |
| 28 | 26.5595 | 2034.6 | 11220.0 |
| 29 | 28.3775 | 1982.9 | 7554.1 |
| 30 | 29.3789 | 1892.3 | 6813.8 |
| 31 | 30.3225 | 2388.9 | 10877.8 |
| 32 | 31.2591 | 3454.6 | 8665.8 |
| 33 | 34.1043 | 3992.9 | 8491.2 |
| 34 | 36.1792 | 977.9 | 2828.7 |

### Example 5: Analysis of RCF Forms 1 and 2 by FT-IR Spectroscopy

For purposes of analysis by Fourier transform infrared spectroscopy (“FT-IR” or “FTIR”), infrared spectra were obtained on a Thermo iS-50 equipped with a single bounce attenuated total reflectance (ATR) attachment with a diamond crystal and DTGS detector. 64 scans with 2 cm⁻¹ resolution were recorded for each spectrum.

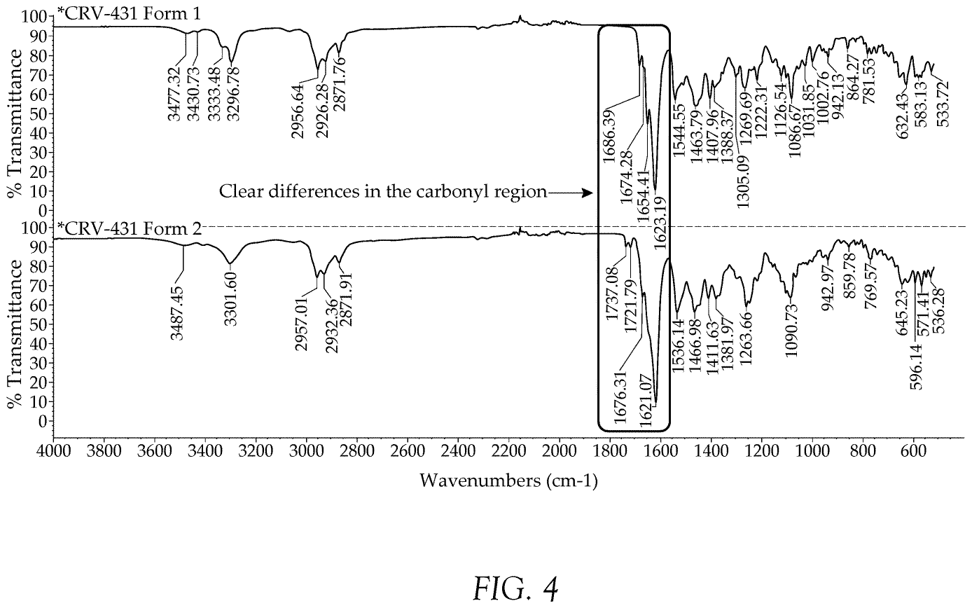

A comparison of the FT-IR spectra of RCF Forms 1 and 2, as depicted in FIG. 4, indicates that there are subtle differences between the two forms across the fingerprint region of the spectra (600-1400 cm⁻¹). Most notably, there are clear differences in the carbonyl stretches for the two forms (1600-1750 cm⁻¹). This is consistent with observations from ssNMR data (see below), and is likely due to differences in hydrogen-bonding synthons present in the respective Forms.

### Example 6: Analysis of RCF Forms 1 and 2 by ¹³C NMR

For purposes of analysis of RCF Forms 1 and 2 by solid state ¹³C nuclear magnetic resonance spectroscopy (“ssNMR”), RCF polymorphs were collected at 23 °C on a Bruker 500 MHz spectrometer equipped with a high-resolution H-F/X BMax 500 DOTY probe. A standard ¹³C cross polarization-magic angle spinning (CP-MAS) experiment with spinning sideband suppression was run, and 10000 scans were collected. Data was processed using MestreNova software.

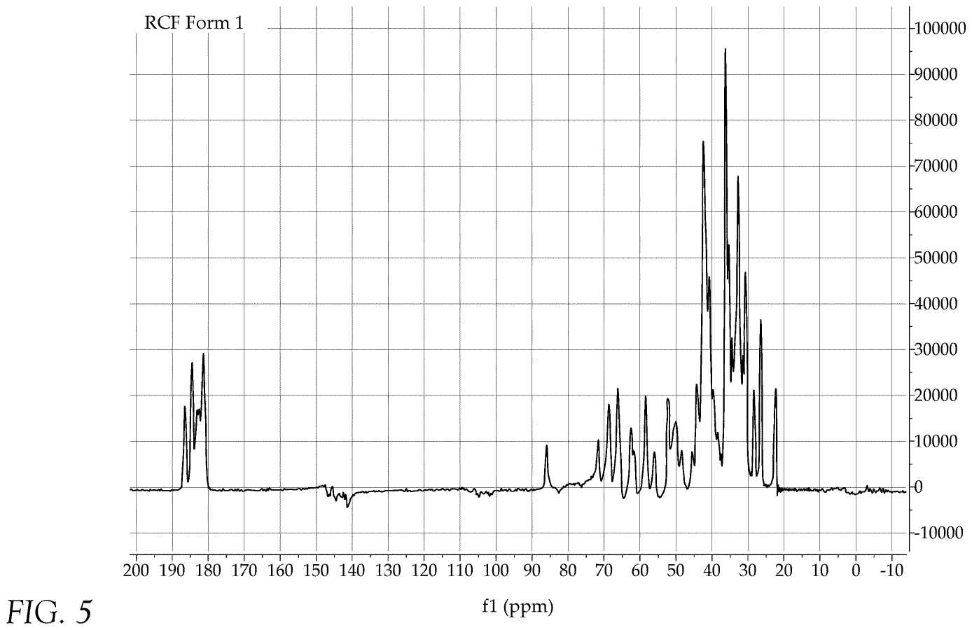

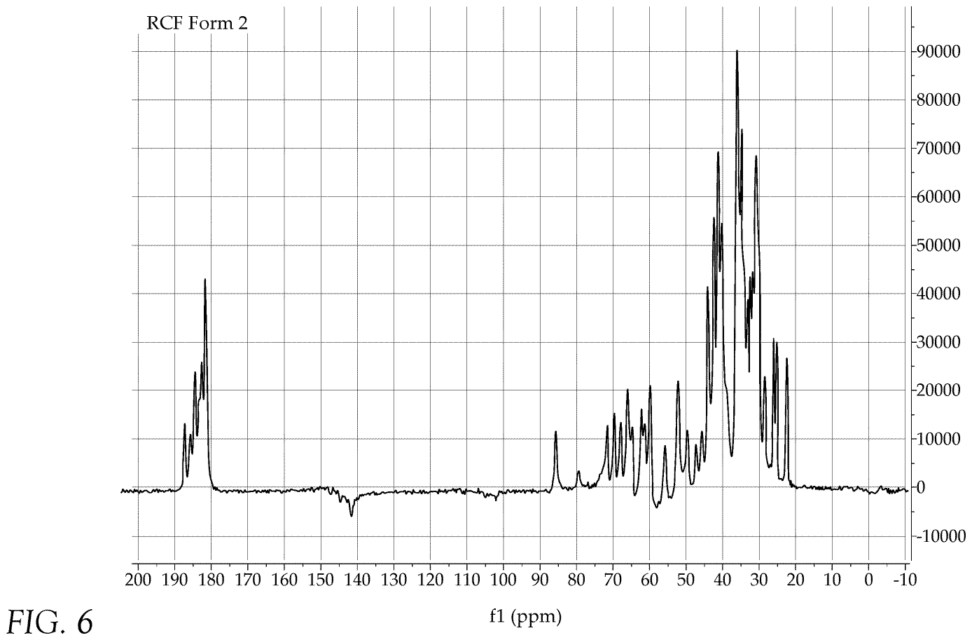

The ssNMR spectra for RCF Forms 1 and 2 are depicted, respectively, in FIG. 5 and FIG. 6. There are small shifts in ¹³C peaks across the spectrum for both forms, but the differences are most pronounced for the carbonyl group (peaks above 180 ppm). Form 2 has additional peaks in the carbonyl region, which is likely due to differences in the hydrogen-bonding synthons involving the carbonyl group. A list of peaks is provided in the following Table 3:

| RCF Form 1 | RCF Form 2 |
|---|---|
| (ppm) | (ppm) |
| 186.54 | 187.29 |
| 184.52 | 185.80 |
| 182.94 | 184.49 |
| 181.38 | 183.51 |
| — | 182.82 |
| — | 181.68 |
| 86.00 | 85.81 |
| — | 79.48 |
| 71.69 | 71.66 |
| 68.74 | 69.64 |
| 66.22 | 67.97 |
| 62.51 | 66.02 |
| 61.58 | 64.79 |
| 58.47 | 62.20 |
| 56.13 | 61.35 |
| 52.31 | 59.89 |
| 51.22 | 55.78 |
| 50.08 | 52.15 |
| 48.39 | 49.64 |
| 45.60 | 47.18 |
| 44.20 | 45.69 |
| 42.45 | 44.04 |
| 41.66 | 42.38 |
| 40.73 | 41.19 |
| 39.52 | 40.21 |
| 38.29 | 38.88 |
| 36.28 | 36.00 |
| 35.46 | 34.79 |
| 34.41 | 34.06 |
| 33.59 | 33.12 |
| 32.69 | 32.35 |
| 31.42 | 30.82 |
| 30.66 | 30.00 |
| 28.34 | 28.50 |
| 26.44 | 25.91 |
| 22.38 | 25.09 |
| — | 22.40 |

### Example 7: Manufacture of SMEDDS-Based Soft Gelatin Capsules

The manufacturing process for soft gelatin capsules is divided into five unit operations:
A. fill material preparation
B. gel preparation
C. encapsulation
D. capsule drying
E. capsule washing

### A. Fill Material Preparation

The active fill mix is done by first weighing off crystalline Form 1 RCF in an isolator and transferring it into an intermediate batch container (IBC) until ready to be added to the main mix.

A side mixture of Glyceryl Monolinoleate and Vitamin E (dl-alpha Tocopherol) is prepared, using a mixer. In parallel, Diethylene glycol monoethyl Ether and Propylene Glycol are added to a primary mixing vessel, and are mixed using an agitator and homogenizer. The RCF is then vacuum transferred to the primary mixing vessel, and the IBC is rinsed with additional Diethylene Glycol Monoethyl Ether. The vessel temperature is then set to 23 °C and the materials are mixed using the agitator and homogenizer for a target of 20 minutes.

The Glyceryl Monolinoleate and Vitamin E side mix is then vacuum transferred to the primary mixing vessel, and the mixture is mixed for not less than 10 minutes. Polyoxyl 40 Hydrogenated Castor Oil is then vacuum transferred with mixing for not less than 10 minutes. The mixture is then de-aerated for a target of 20 minutes.

After de-aeration, the vacuum is re-established and then Dehydrated Ethanol is vacuum transferred into the primary mixing vessel. The mix is homogenized for not less than 90 minutes. After 60 minutes, a fill sample is taken via a sampling attachment to check if the RCF is fully dissolved, via visual inspection.

After not less than 90 minutes and once the mixture is confirmed to be clear of undissolved RCF, the mix is then discharged into the receiver; and the receiver is blanketed with Nitrogen.

### B and C. Gel Preparation & Encapsulation

A standard pharmaceutical gel material is prepared prior to encapsulation. Fill material is encapsulated inside the gel material in order to form the soft gelatin capsules. Throughout the encapsulation process, samples are pulled and tested for fill weight, shell weight, seal thickness and print inspection (if necessary).

### D. Capsule Drying

Capsules are dried immediately after encapsulation in a series of tumble dryer baskets. Capsules are then transferred to shallow trays which are stacked and placed inside drying tunnels operating at specified temperature and humidity conditions. Throughout the drying process, capsules are tested for hardness. Capsules are dried within a specified hardness range in order to produce the final dosage form.

### E. Capsule Washing

Fractionated coconut oil and a fractionated coconut oil/lecithin blend (0.1%) used to lubricate the gelatin ribbon on the encapsulation machine are removed from the capsules during a washing step using a liquid, water-free phospholipid concentrate, preferably phosphatidylcholine in medium-chain triglyceride, content≥53.0% (available as Phosal 53 MCT, Lipoid GmbH, Ludwigshafen, Germany) and denatured ethanol which is sprayed directly onto capsules. Wash solvent is removed by tumble drying.

Having described the invention with reference to certain preferred embodiments, other embodiments will become apparent to one skilled in the art from consideration of the specification. It will be apparent to those skilled in the art that many modifications, both to materials and methods, may be practiced without departing from the scope of the invention.

We claim:

1. A solid state form of Rencofilstat designated as Form 1, characterized by data selected from one or more of the following:
(a) a powder X-ray diffraction (PXRD) pattern having the following peaks when measured at an angle of 2-theta±0.2 degrees 2-theta:

| | Position | | |
|---|---|---|---|
| # | 2θ Value | Intensity | Height |
| 1 | 4.7083 | 110408.4 | 506201.8 |
| 2 | 5.5849 | 4938.0 | 19820.2 |
| 3 | 6.9325 | 23830.6 | 97647.3 |
| 4 | 7.3994 | 31711.6 | 146608.3 |
| 5 | 8.6831 | 44562.8 | 179637.7 |
| 6 | 9.0191 | 24454.4 | 127945.9 |
| 7 | 9.5021 | 13383.6 | 73720.9 |
| 8 | 9.9659 | 32574.3 | 105011.0 |
| 9 | 10.2239 | 40704.6 | 224840.2 |
| 10 | 10.8055 | 4394.7 | 21434.1 |
| 11 | 11.2675 | 29302.0 | 128555.3 |
| 12 | 12.4411 | 20228.9 | 73141.2 |
| 13 | 13.3245 | 7920.7 | 45356.6 |
| 14 | 14.3006 | 19463.1 | 81718.0 |
| 15 | 14.7921 | 20015.4 | 99330.9 |
| 16 | 15.2066 | 5979.3 | 27102.5 |
| 17 | 15.7326 | 35689.7 | 142788.1 |
| 18 | 16.1205 | 30936.2 | 167994.0 |
| 19 | 16.9995 | 56657.4 | 182244.2 |
| 20 | 17.3235 | 13650.7 | 76633.3 |
| 21 | 17.6441 | 44849.0 | 208985.8 |
| 22 | 18.2004 | 2024.8 | 19622.8 |
| 23 | 18.9752 | 10743.3 | 49581.2 |
| 24 | 19.5894 | 52059.2 | 170752.0 |
| 25 | 20.0000 | 10473.1 | 43527.3 |
| 26 | 20.6846 | 21595.7 | 59901.1 |
| 27 | 21.1045 | 13355.2 | 48305.6 |
| 28 | 21.5370 | 9929.7 | 46979.1 |
| 29 | 21.9448 | 8187.3 | 38250.4 |
| 30 | 22.8054 | 51297.4 | 130002.2 |
| 31 | 24.0205 | 41704.5 | 153171.9 |
| 32 | 25.0603 | 29177.3 | 88303.9 |
| 33 | 25.6479 | 6381.3 | 26373.9 |
| 34 | 26.2237 | 8467.7 | 41999.4 |
| 35 | 27.0195 | 19906.9 | 74652.4 |
| 36 | 27.9666 | 19348.7 | 70994.9 |
| 37 | 29.4712 | 12866.7 | 55353.3 |
| 38 | 30.3381 | 6590.5 | 23877.9 |
| 39 | 31.0550 | 3514.6 | 12175.9 |
| 40 | 31.9574 | 11339.0 | 21506.4 |
| 41 | 32.7767 | 3029.2 | 12873.4 |
| 42 | 34.0085 | 9293.7 | 26166.1 |
| 43 | 35.1488 | 7014.5 | 34897.0 |
| 44 | 36.3905 | 3607.4 | 16735.8 |
| 45 | 36.9099 | 1433.2 | 9239.4 |
| 46 | 38.7824 | 4549.6 | 7702.9 |

(b) a PXRD pattern substantially as depicted in FIG. 2;
(c) a solid state ¹³C NMR spectrum having characteristic peaks at: 186.54, 184.52 and 181.38 ppm±0.2 ppm;
(d) a solid state ¹³C NMR spectrum having the following chemical shift absolute differences from reference peak at 86.00 ppm±1 ppm: 100.54, 98.52 and 95.38 ppm±0.1 ppm;
(e) a solid state ¹³C NMR spectrum having peaks at: 186.54, 184.52, 182.94, 181.38, 86.00, 71.69, 68.74, 66.22, 62.51, 61.58, 58.47, 56.13, 52.31, 51.22, 50.08, 48.39, 45.60, 44.20, 42.45, 41.66, 40.73, 39.52, 38.29, 36.28, 35.46, 34.41, 33.59, 32.69, 31.42, 30.66, 28.34, 26.44, and 22.38 ppm±0.2 ppm;
(f) a solid state ¹³C NMR spectrum substantially as depicted in FIG. 5;
(g) an FT-IR spectrum having peaks at: 533.72, 583.13, 632.43, 781.53, 864.27, 942.13, 1002.76, 1031.85, 1086.67, 1126.54, 1222.31, 1269.69, 1305.09, 1388.37, 1407.96, 1463.79, 1544.55, 1623.19, 1654.41, 1674.28, 1686.39, 2871.76, 2926.28, 2956.64, 3296.78, 3333.48, 3430.73, and 3477.32 cm⁻¹±4 cm⁻¹; and
(h) a combination of any two or more of (a)-(g).

2. The solid state form of Rencofilstat according to claim 1, characterized by a PXRD pattern having peaks at: 4.7083, 8.6831, 10.2239, 16.9995 and 17.6441 degrees 2-theta±0.2 degrees 2-theta, and also having any one, two, three, four or five additional peaks at: 7.3994, 15.7326, 16.1205, 19.5894 and 24.0205 degrees 2-theta±0.2 degrees 2-theta.

3. The solid state form of Rencofilstat according to claim 1, characterized by data selected from one or more of the following:
(a) a PXRD pattern having the following peaks when measured at an angle of 2-theta±0.2 degrees 2-theta:

| | Position | | |
|---|---|---|---|
| # | 2θ Value | Intensity | Height |
| 1 | 4.7083 | 110408.4 | 506201.8 |
| 2 | 5.5849 | 4938.0 | 19820.2 |
| 3 | 6.9325 | 23830.6 | 97647.3 |
| 4 | 7.3994 | 31711.6 | 146608.3 |
| 5 | 8.6831 | 44562.8 | 179637.7 |
| 6 | 9.0191 | 24454.4 | 127945.9 |
| 7 | 9.5021 | 13383.6 | 73720.9 |
| 8 | 9.9659 | 32574.3 | 105011.0 |
| 9 | 10.2239 | 40704.6 | 224840.2 |
| 10 | 10.8055 | 4394.7 | 21434.1 |
| 11 | 11.2675 | 29302.0 | 128555.3 |
| 12 | 12.4411 | 20228.9 | 73141.2 |
| 13 | 13.3245 | 7920.7 | 45356.6 |
| 14 | 14.3006 | 19463.1 | 81718.0 |
| 15 | 14.7921 | 20015.4 | 99330.9 |
| 16 | 15.2066 | 5979.3 | 27102.5 |
| 17 | 15.7326 | 35689.7 | 142788.1 |
| 18 | 16.1205 | 30936.2 | 167994.0 |
| 19 | 16.9995 | 56657.4 | 182244.2 |
| 20 | 17.3235 | 13650.7 | 76633.3 |
| 21 | 17.6441 | 44849.0 | 208985.8 |
| 22 | 18.2004 | 2024.8 | 19622.8 |
| 23 | 18.9752 | 10743.3 | 49581.2 |
| 24 | 19.5894 | 52059.2 | 170752.0 |
| 25 | 20.0000 | 10473.1 | 43527.3 |
| 26 | 20.6846 | 21595.7 | 59901.1 |
| 27 | 21.1045 | 13355.2 | 48305.6 |
| 28 | 21.5370 | 9929.7 | 46979.1 |
| 29 | 21.9448 | 8187.3 | 38250.4 |
| 30 | 22.8054 | 51297.4 | 130002.2 |
| 31 | 24.0205 | 41704.5 | 153171.9 |
| 32 | 25.0603 | 29177.3 | 88303.9 |
| 33 | 25.6479 | 6381.3 | 26373.9 |
| 34 | 26.2237 | 8467.7 | 41999.4 |
| 35 | 27.0195 | 19906.9 | 74652.4 |
| 36 | 27.9666 | 19348.7 | 70994.9 |
| 37 | 29.4712 | 12866.7 | 55353.3 |
| 38 | 30.3381 | 6590.5 | 23877.9 |
| 39 | 31.0550 | 3514.6 | 12175.9 |
| 40 | 31.9574 | 11339.0 | 21506.4 |
| 41 | 32.7767 | 3029.2 | 12873.4 |
| 42 | 34.0085 | 9293.7 | 26166.1 |
| 43 | 35.1488 | 7014.5 | 34897.0 |
| 44 | 36.3905 | 3607.4 | 16735.8 |
| 45 | 36.9099 | 1433.2 | 9239.4 |
| 46 | 38.7824 | 4549.6 | 7702.9 |

(b) a PXRD pattern substantially as depicted in FIG. 2; and
(c) a combination of (a) and (b).

4. The solid state form of Rencofilstat according to claim 3, characterized by a PXRD pattern having peaks at: 4.7083, 8.6831, 10.2239, 16.9995 and 17.6441 degrees 2-theta±0.2 degrees 2-theta, and also having any one, two, three, four or five additional peaks at: 7.3994, 15.7326, 16.1205, 19.5894 and 24.0205 degrees 2-theta±0.2 degrees 2-theta.

5. A solid state form of Rencofilstat designated as Form 2, characterized by data selected from one or more of the following:
(a) a powder X-ray diffraction (PXRD) pattern having the following peaks when measured at an angle of 2-theta±0.2 degrees 2-theta:

| | Position | | |
|---|---|---|---|
| # | 2θ Value | Intensity | Height |
| 1 | 4.6736 | 19955.5 | 99826.7 |
| 2 | 5.8747 | 8398.2 | 38897.8 |
| 3 | 7.6125 | 14330.3 | 80845.4 |
| 4 | 7.9550 | 16095.2 | 101606.1 |
| 5 | 8.4823 | 5571.0 | 29324.1 |
| 6 | 9.4833 | 36325.4 | 183229.2 |
| 7 | 10.6611 | 7462.2 | 36808.3 |
| 8 | 11.8725 | 22234.4 | 109115.8 |
| 9 | 13.5826 | 3056.5 | 14816.2 |
| 10 | 14.9173 | 4586.7 | 29826.3 |
| 11 | 15.2227 | 11555.9 | 68004.3 |
| 12 | 16.0121 | 3415.7 | 19811.9 |
| 13 | 16.3762 | 2050.1 | 12136.3 |
| 14 | 16.8084 | 5000.0 | 20991.0 |
| 15 | 17.5943 | 19701.4 | 96090.9 |
| 16 | 18.1623 | 9297.5 | 38272.9 |
| 17 | 18.7169 | 3070.0 | 20037.7 |
| 18 | 19.1112 | 6916.9 | 37519.0 |
| 19 | 20.2712 | 4754.8 | 25291.7 |
| 20 | 20.4733 | 2900.9 | 20181.2 |
| 21 | 20.8856 | 3097.6 | 16085.0 |
| 22 | 21.9116 | 6018.9 | 14049.2 |
| 23 | 22.5662 | 5268.3 | 13773.0 |
| 24 | 23.5071 | 3218.8 | 11820.8 |
| 25 | 23.8611 | 5452.1 | 25641.9 |
| 26 | 24.4246 | 3720.4 | 17285.2 |
| 27 | 25.1597 | 6319.7 | 20122.7 |
| 28 | 26.5595 | 2034.6 | 11220.0 |
| 29 | 28.3775 | 1982.9 | 7554.1 |
| 30 | 29.3789 | 1892.3 | 6813.8 |
| 31 | 30.3225 | 2388.9 | 10877.8 |
| 32 | 31.2591 | 3454.6 | 8665.8 |
| 33 | 34.1043 | 3992.9 | 8491.2 |
| 34 | 36.1792 | 977.9 | 2828.7 |

(b) a PXRD pattern substantially as depicted in FIG. 3;
(c) a solid state ¹³C NMR spectrum having characteristic peaks at: 187.29, 184.49, 182.82 and 181.68 ppm±0.2 ppm;
(d) a solid state ¹³C NMR spectrum having the following chemical shift absolute differences from reference peak at 85.81 ppm #1 ppm: 101.48, 98.68, 97.01 and 95.87 ppm±0.1 ppm;
(e) a solid state ¹³C NMR spectrum having peaks at: 187.29, 185.80, 184.49, 183.51, 182.82, 181.68, 85.81, 79.48, 71.66, 69.64, 67.97, 66.02, 64.79, 62.20, 61.35, 59.89, 55.78, 52.15, 49.64, 47.18, 45.69, 44.04, 42.38, 41.19, 40.21, 38.88, 36.00, 34.79, 34.06, 33.12, 32.35, 30.82, 30.00, 28.50, 25.91, 25.09, and 22.40 ppm±0.2 ppm;
(f) a solid state ¹³C NMR spectrum substantially as depicted in FIG. 6;
(g) an FT-IR spectrum having peaks at: 536.28, 571.41, 596.14, 645.23, 769.57, 859.78, 942.97, 1090.73, 1263.66, 1381.97, 1411.63, 1466.98, 1536.14, 1621.07, 1676.31, 1721.79, 1737.08, 2871.91, 2932.36, 2957.01, 3301.60, and 3487.45 cm⁻¹±4 cm⁻¹; and
(h) a combination of any two or more of (a)-(g).

6. The solid state form of Rencofilstat according to claim 5, characterized by a PXRD pattern having peaks at: 4.6736, 7.9550, 9.4833, 11.8725 and 17.5943 degrees 2-theta±0.2 degrees 2-theta, and also having any one, two, three, four or five additional peaks at: 5.8747, 7.6125, 15.2227, 18.1623, and 19.1112 degrees 2-theta±0.2 degrees 2-theta.

7. The solid state form of Rencofilstat according to claim 5, characterized by data selected from one or more of the following:
(a) a powder X-ray diffraction (PXRD) pattern having the following peaks when measured at an angle of 2-theta±0.2 degrees 2-theta:

| | Position | | |
|---|---|---|---|
| # | 2θ Value | Intensity | Height |
| 1 | 4.6736 | 19955.5 | 99826.7 |
| 2 | 5.8747 | 8398.2 | 38897.8 |
| 3 | 7.6125 | 14330.3 | 80845.4 |
| 4 | 7.9550 | 16095.2 | 101606.1 |
| 5 | 8.4823 | 5571.0 | 29324.1 |
| 6 | 9.4833 | 36325.4 | 183229.2 |
| 7 | 10.6611 | 7462.2 | 36808.3 |
| 8 | 11.8725 | 22234.4 | 109115.8 |
| 9 | 13.5826 | 3056.5 | 14816.2 |
| 10 | 14.9173 | 4586.7 | 29826.3 |
| 11 | 15.2227 | 11555.9 | 68004.3 |
| 12 | 16.0121 | 3415.7 | 19811.9 |
| 13 | 16.3762 | 2050.1 | 12136.3 |
| 14 | 16.8084 | 5000.0 | 20991.0 |
| 15 | 17.5943 | 19701.4 | 96090.9 |
| 16 | 18.1623 | 9297.5 | 38272.9 |
| 17 | 18.7169 | 3070.0 | 20037.7 |
| 18 | 19.1112 | 6916.9 | 37519.0 |
| 19 | 20.2712 | 4754.8 | 25291.7 |
| 20 | 20.4733 | 2900.9 | 20181.2 |
| 21 | 20.8856 | 3097.6 | 16085.0 |
| 22 | 21.9116 | 6018.9 | 14049.2 |
| 23 | 22.5662 | 5268.3 | 13773.0 |
| 24 | 23.5071 | 3218.8 | 11820.8 |
| 25 | 23.8611 | 5452.1 | 25641.9 |
| 26 | 24.4246 | 3720.4 | 17285.2 |
| 27 | 25.1597 | 6319.7 | 20122.7 |
| 28 | 26.5595 | 2034.6 | 11220.0 |
| 29 | 28.3775 | 1982.9 | 7554.1 |
| 30 | 29.3789 | 1892.3 | 6813.8 |
| 31 | 30.3225 | 2388.9 | 10877.8 |
| 32 | 31.2591 | 3454.6 | 8665.8 |
| 33 | 34.1043 | 3992.9 | 8491.2 |
| 34 | 36.1792 | 977.9 | 2828.7 |

(b) a PXRD pattern substantially as depicted in FIG. 3; and
(c) a combination of (a) and (b).

8. The solid state form of Rencofilstat according to claim 7, characterized by a PXRD pattern having peaks at: 4.6736, 7.9550, 9.4833, 11.8725 and 17.5943 degrees 2-theta±0.2 degrees 2-theta, and also having any one, two, three, four or five additional peaks at: 5.8747, 7.6125, 15.2227, 18.1623, and 19.1112 degrees 2-theta±0.2 degrees 2-theta.

9. The solid state form of Rencofilstat according to claim 1, containing: 20% or less, 10% or less, 5% or less, 2% or less, 1% or less, 0.5% or less, or about 0% of any other solid state form of Rencofilstat.

10. The solid state form of Rencofilstat according to claim 5, containing: 20% or less, 10% or less, 5% or less, 2% or less, 1% or less, 0.5% or less, or about 0% of any other solid state form of Rencofilstat.

11. A pharmaceutical composition comprising a solid state form of Rencofilstat according to claim 1.

12. A pharmaceutical composition comprising a solid state form of Rencofilstat according to claim 5.

13. A pharmaceutical formulation comprising a solid state form of Rencofilstat according to claim 1 and at least one pharmaceutically acceptable excipient.

14. A pharmaceutical formulation comprising a solid state form of Rencofilstat according to claim 5 and at least one pharmaceutically acceptable excipient.

15. A method of manufacturing a pharmaceutical dosage form based on a self-microemulsifying drug delivery system (SMEDDS), the method comprising:
solubilizing solid state Form 1 Rencofilstat in a mixture of Vitamin E, glyceryl monolinoleate, propylene glycol, diethylene glycol monoethyl ether, ethanol, and polyoxyl castor oil, wherein the Vitamin E, glyceryl monolinoleate, propylene glycol, diethylene glycol monoethyl ether, ethanol, and polyoxyl castor oil are at a weight ratio, respectively, of about (0.75-1.5)/(0.5-2)/(2-5)/(2-5)/(2-2.4)/(4-8).

16. A method of manufacturing a pharmaceutical dosage form based on a spray dried dispersion, the method comprising:
(i) providing a mixture of solid state Form 1 Rencofilstat with a pharmaceutically acceptable polymer;
(ii) dissolving the mixture in an organic solvent;
(iii) forming liquid droplets of the dissolved mixture;
(iv) spraying the liquid droplets onto a receiving surface; and
(v) drying the sprayed mixture to remove residual solvent.
